(19) United States
(12) Patent Application Publication
(10) Pub. No.: US 2021/0245007 A1
(43) Pub. Date: Aug. 12, 2021

(54) SYSTEM FOR ANALYZING A PEDALLING TECHNIQUE OF A CYCLIST AND ASSOCIATED METHODS

(71) Applicant: ZHOR TECH, Nancy (FR)

(72) Inventor: Karim OUMNIA, Nancy (FR)

(21) Appl. No.: 17/054,558

(22) PCT Filed: May 13, 2019
(86) PCT No.: PCT/FR2019/051080

§ 371 Date: Nov. 11, 2020

(30) Foreign Application Priority Data
May 11, 2018 (FR) 1870552

(51) Int. Cl. A63B 24/00; B62J 45/421; G01P 15/08; G01C 19/00; G01P 15/18; G16H 40/67; G16H 50/30; A43B 3/00; A43B 5/14

(52) U.S. Cl. CPC A63B 24/0062; B62J 45/421; G01P 15/08; G01C 19/00; G01P 15/18; G16H 50/70; G16H 50/30; A43B 3/0005; A43B 5/14; A63B 2220/40; A63B 2220/836; G16H 40/67

(57) ABSTRACT

A device for analyzing and quantifying the pedaling technique of a user, based on a pair of soles, each sole comprising a compact and miniaturized electronic box, which comprises an electronic board containing an inertial platform consisting in particular of an accelerometer and a gyroscope, used to collect and process biomechanical data, the two boxes being adapted to communicate with each other and with an external terminal in order to provide the user with general information on his/her pedaling activity.

### TECHNICAL AREA

The invention relates to the field of shoes and more generally to that of footwear. The present invention relates more particularly to a system for analyzing the pedaling technique of a cyclist.

More precisely, it is a biomechanical data measurement system consisting of two boxes, each one arranged in each of the two shoe soles of a same pair, designed to collect and use information on the pedaling technique of a user.

### PRIOR ART

Shoes, and more specifically soles, whether inside or outside, essentially have the original role of protecting the foot from the ground. Their shape varies according to fashion and its vagaries to make room for a multitude of by-products and functions.

Shoes can be for relaxation, formal, sports, medical, professional, or simply recreational use. Thus, a shoe consists mainly of, on the one hand, a sole, the lower part which protects the sole of the feet, more or less raised at the back by the heel, and, on the other hand, the upper, the upper part which envelops the foot. It can be limited to the ankle or it can be a high shoe. The sole can be made in two parts. An upper sole layer in direct contact with the user's foot and a lower sole layer in direct contact with the ground or more generally the outside environment. A shoe may also include a removable insole. In this particular case, this sole also consists of at least one upper sole layer and one lower sole layer.

New technologies have been accompanying new needs and the world of footwear has been part of this movement. The development of electronics has led to the appearance of so-called connected soles and shoes, which have a wide range of functions. Very generally, these connected soles or shoes can be autonomous and contain a rechargeable battery. They can be connected to an external terminal by a wired system or wireless connection.

Among the functions offered by existing connected soles or shoes, we can mention the function of heating the foot to one or more given temperatures, determined by the manufacturer; we can also mention those, described in document US2017/0188950, equipped with pressure sensors and an accelerometer and which allow a smartphone connected via Bluetooth to deliver statistics on the physical activities of their wearer such as the number of steps taken.

More specifically, as regards the pedaling activity, the inventions listed all relate to devices operating from the bicycle, such as WO 2010000369 which allows the output power of a cyclist to be measured: this is a force sensor embedded in a bicycle cleat, and bolted to the cyclist's shoe. This invention, which uses an accelerometer and pressure sensors, remains a device attached to the bicycle and needs to be moved when the bicycle is changed. Moreover, this device only allows for data collection after the pedaling activity.

WO2010014242 and EP1637445 suffer from the same criticism, so that despite the variety of technological solutions offered, no user has access to insoles that allow him/her to monitor in real time his/her pedaling activity from data collected directly from his/her feet activity. Thus, there is a need for new systems for analyzing the pedaling technique of a cyclist.

### TECHNICAL PROBLEM

The invention aims to overcome the disadvantages of the prior art. In particular, the invention aims at providing a system for analyzing the pedaling technique of a cyclist, said system being reliable, robust, and allowing the activity of the cyclist to be monitored in real time and over a longer period of time thanks in particular to an improved autonomy.

The invention, furthermore, aims at providing a method for analyzing the pedaling technique, where said method can be implemented in real time and essentially at the sole. This method allows at least one biomechanical parameter representative of the user's pedaling technique to be established and an alphanumeric value representative of the evolution of the cyclist's pedaling technique to be calculated, in a quick, simple way, not necessarily requiring the intervention of a specialist in the field of sport.

### BRIEF DESCRIPTION OF THE INVENTION

In particular, the invention relates to a system for quantifying the pedaling of a user to obtain a value representative of the pedaling technique of said user, including a pair of soles, the soles, constituting said pair of soles, each including an electronic box, each electronic box comprising:
an inertial platform configured to generate a data set on the pedaling technique of a user of the pair of soles,
a data processing module configured to transform the generated data set into at least one biomechanical parameter and determine the evolution of these biomechanical parameters,
a data storage module configured to store the at least one biomechanical parameter, a first communication means configured so that the electronic box of at least one of the soles is adapted to transmit the at least one biomechanical parameter and/or the value representative of the evolution of the user's pedaling technique to the external terminal, and
a power source.

Alternatively, the invention relates to a system for analyzing and quantifying the pedaling of a user, characterized in that it includes two electronic boxes, adapted to be integrated into a pair of soles, a first box being adapted to be integrated into a first sole and a second box being adapted to be integrated into a second sole, each box comprising:
an inertial platform configured to generate a data set on the pedaling technique of a user of the pair of soles,
a data processing module configured to, on the one hand, process the set of data collected according to predefined algorithms and transform this set into at least one biomechanical parameter and, on the other hand, determine the evolution of these biomechanical parameters over time,
a data storage module configured to store the information generated by the processing module,
a first communication means configured so that the electronic box of at least one of the soles is adapted to transmit the information generated on the user's pedaling technique to an external terminal and/or to the other box of the other sole, and
a power source.

The advantage of this invention is that it makes it possible to have an electronic box containing all the electronic components necessary for autonomous operation, such as all the sensors, including calculation modules and an energy source. This also increases the robustness of the system. This box can advantageously be unique, compact, and miniaturized.

Moreover, contrary to the systems proposed in the prior art, calculation is carried out here at the sole via a data processing module which can correspond to the firmware (“firmware” in Anglo-Saxon terminology) of an electronic board. In this way, the data is processed almost in real time at the electronic box, compared and can then be transferred for visualization on an external terminal. Such a system allows the load on the memory of the storage module to be reduced and can therefore increase the autonomy of the system.

Each electronic box further includes a second communication means configured so that the electronic box of a first sole is adapted to communicate with the electronic box of a second sole, and so that at least one of the data processing modules is configured to transform data sets generated from the two soles constituting the pair of soles into at least one biomechanical parameter relating to the pedaling technique. Such a configuration allows more relevant biomechanical parameters to be produced in real time for the study of the user's pedaling technique.

The data processing modules communicating with each other are adapted, on the one hand, to calculate an asymmetry between the biomechanical parameters of the right leg with respect to the biomechanical parameters of the left leg and, on the other hand, to calculate the variability of the biomechanical parameters associated with one leg or both legs. Such information can be of great interest when quantifying the users' pedaling technique.

Each data processing module is adapted to establish a pedaling technique profile of the user from biomechanical parameters such as foot angle, efficiency of the push and pull phase, foot position, cadence, rotational speed, and power.

The transformation by the data processing module comprises the segmentation of the data into a plurality of pedaling phases. Such segmentation allows for a better analysis of the user's pedaling. In earlier devices, it is not performed within the sole as is the case in the present invention.

Each electronic box further includes other sensors, in particular a magnetometer, a barometer, an altimeter, force and pressure sensors.

The calculation of the pedaling power is carried out following the combination of inertial and pressure/force data.

In addition, each of the two boxes is designed to be integrated into the pedal of a bicycle, or into the cleat of the pedal of a bicycle.

The invention further relates to a method for quantifying the pedaling technique of a user implementing a quantification system including a pair of soles and an external terminal, the soles constituting said pair of soles, each including an electronic box, each electronic box comprising an inertial platform, a data processing module, a data storage module, a first communication means, and a power source, said method including the following steps:
Generating, by the inertial platform, a set of data on the pedaling technique of the user of the pair of soles,
Transforming, by the data processing module, the generated data set into at least one biomechanical parameter, and comparing these parameters,
Storing, by the data storage module, at least one biomechanical parameter,
Transmitting, by a first communication means of at least one of the soles, the at least one biomechanical parameter and/or the value representative of the evolution of the individual's pedaling to the external terminal.

### BRIEF DESCRIPTION OF THE DRAWINGS

FIG. 1 shows a longitudinal cross-sectional top view of two soles, each containing a cavity which will give way to a box, each of the antennas of the boxes being located on the outer edge facing each foot, according to one embodiment of the invention.

FIG. 2 shows an open electronic box as seen from above comprising in particular an electronic board, a rechargeable battery, a connector, and an antenna.

FIG. 3 shows an electronic box, in exploded and profile section view, comprising in particular a rechargeable battery, an electronic board, as well as a two-part outer casing.

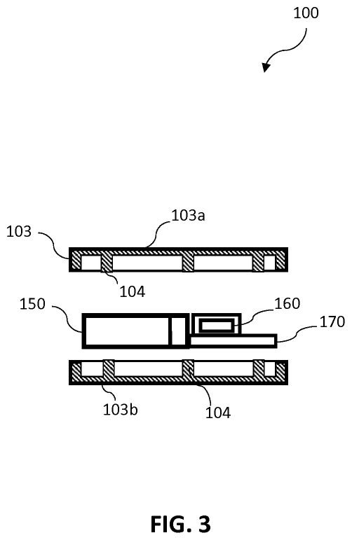

### DESCRIPTION OF THE INVENTION

In order to pursue its objective, the invention consists of an autonomous technology inserted into a compact and miniaturized box of a few grams, which is inserted into each of the two soles of a same pair.

The system 1 according to the invention includes a pair 10 of soles and an external terminal 20.

The soles 11, 12 usable within the context of the system 1 according to the invention may, for example, correspond to outsoles or insoles, of shoes. These soles can be removable or permanently integrated into the sole assembly of the shoes. Preferably, the soles are removable insoles.

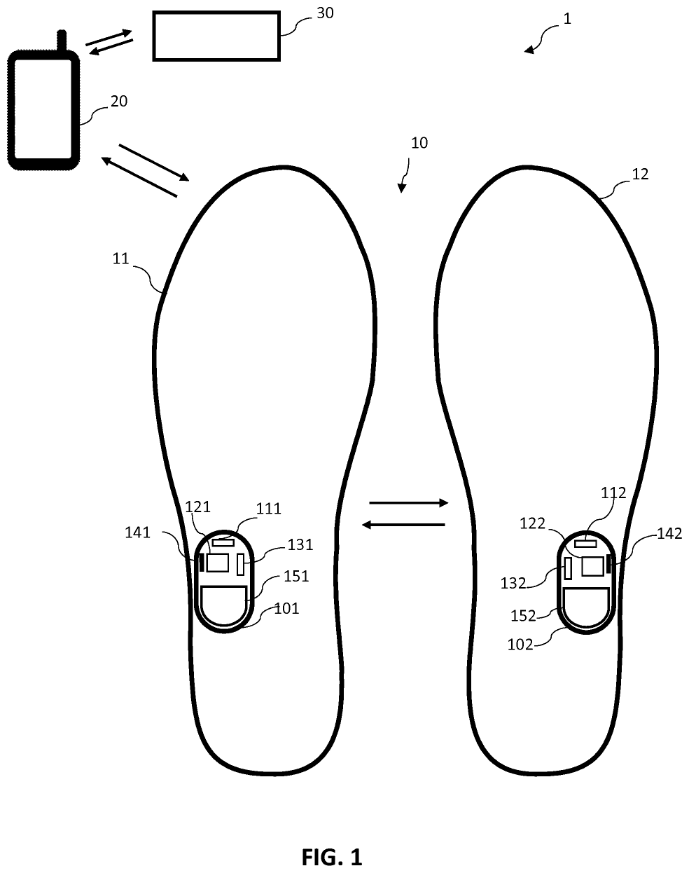

Classically, the soles 11, 12 constituting said pair 10 of soles, each include an electronic box 100, 101, 102. As shown in FIG. 1, the electronic box 101, 102 is preferably positioned at a midsole portion.

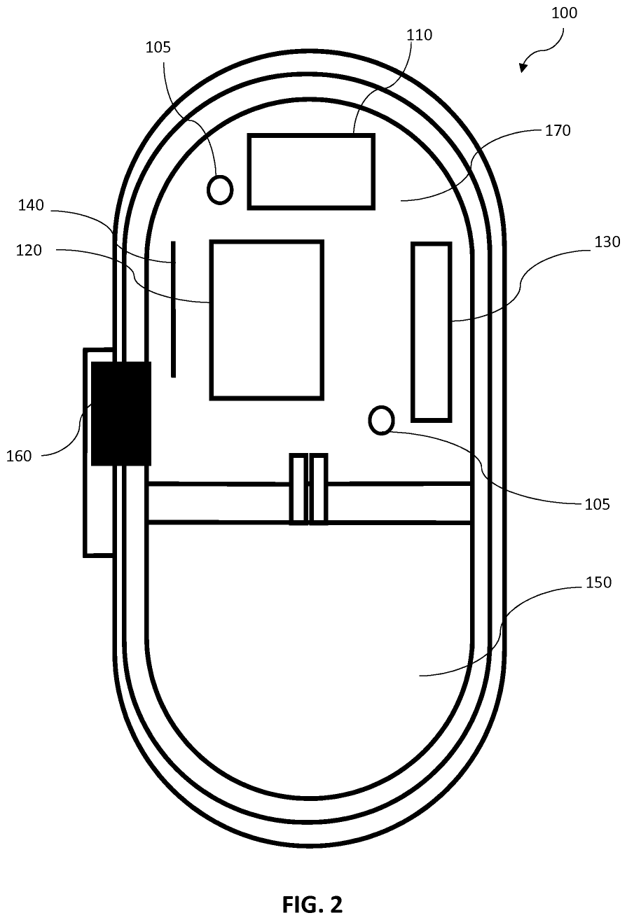

An electronic box 100 according to the invention is detailed in FIG. 2. Weighing only a few grams and being small in size, this electronic box 100 fits into any insole and/or outsole in a space-saving manner.

Thus, each electronic box is preferably light and weighs less than 10 grams, preferably less than 8 grams, and more preferably less than 6 grams. In addition, it may have a thickness of less than 5 mm, preferably less than 4 mm, and more preferably less than 3 mm. This allows it to be easily integrated into a shoe/sole without altering user comfort in his/her shoe. Finally, each electronic box has less than 5 cm² of surface area on its largest face, preferably less than 4 cm², and more preferably less than 3 cm².

Each electronic box advantageously integrates support pillars or pads 104 in order to reinforce its solidity, preferably one pad/cm² to withstand the pressures and impact forces of the movements of the foot. Inserting such pads allows the box to better withstand a person's weight.

Preferably with a rounded shape to increase its mechanical resistance, it must be assembled in such a way as to maintain a perfect seal and make the inside containing the electronic board and the power source protected from humidity and dust.

Thus, preferably, the electronic box 100 according to the invention includes at least two support pads 104, more preferably at least three support pads 104, and even more preferably at least four support pads 104.

Advantageously, the electronic box 100 includes an electronic board with at least one opening 105 allowing the passage of at least one support pad 104, preferably at least two openings 105.

In addition, in order to further increase the robustness of the system, each electronic box includes a shock-absorbing material such as polymer foam (for example polyurethane, polyether). According to one embodiment, the shock-absorbing material has a density between 20 kg/m³ and 50 kg/m³. Such a protective foam layer also allows the board to be insulated from vibrations and humidity.

According to one embodiment of the invention, the electronic board is inserted into a compartment of the box specially designed to receive it.

According to another embodiment, the electronic box 100 is formed by the encapsulation of its components. For example, the encapsulation can take the form of an encapsulating coating or a resin (for example silicone, epoxy, polyurethane). The encapsulation of all components (for example inertial platform, processing module . . . ) offers good insulation and thus combines good electrical properties with excellent mechanical protection.

The electronic box according to the invention is provided with an inertial platform 110, 111, 112 configured to generate a set of data on the pedaling technique of a user of the pair 10 of soles.

During a user's pedaling session, the inertial platform 110 acquires signals representative of a movement parameter (acceleration and/or speed, for example angular velocity) of the foot along the X, Y, Z axes. In addition, this data can then be processed to generate at least one acceleration signal. The inertial platform consists for example of at least one accelerometer and one gyroscope. Preferably, it includes several accelerometers and gyroscopes.

The electronic box may also include one or more magnetometers in order to acquire three additional raw signals corresponding to the values of magnetic fields in three dimensions.

In addition, each electronic box can include other sensors, in particular an inclinometer, a barometer, and an altimeter for increased accuracy.

In addition, the electronic box according to the invention includes a data processing module 120, 121, 122 configured to transform the set of data generated using predefined algorithms.

This processing module allows for a 3D analysis of the pedaling activity, and more particularly everything that will be qualified as the pedaling technique, from the data collected by the inertial platform and any additional sensors placed in the sole.

This processing module can be used to generate biomechanical parameters on how the user pedals. Advantageously, the data processing module 120 is adapted to transform the data set into at least one pedaling biomechanical parameter, said pedaling biomechanical parameter preferably being selected from: foot angle, efficiency of the push and pull phase, foot position, cadence, speed, or power.

Advantageously, the processing module can compare the user's biomechanical parameters to determine the evolution over time of his/her pedaling technique.

In addition, the transformation by the data processing module may advantageously comprise the segmentation of the pedaling into a plurality of phases. Preferably, the data processing module is adapted to segment the pedaling in two phases: push phase and pull phase.

The information generated will then be transmitted to the second box by transmitting signals which can be of the Bluetooth type.

When an electronic box is not able to communicate in real time with the other box and/or with the terminal, it stores the collected information and will transmit it in delayed mode when the exchange is possible again. This delayed transmission of the collected data is made possible using the storage capacity each of the electronic boxes is provided with.

Thus, the electronic box according to the invention includes a data storage module 130, 131, 132, configured to store at least part of the transformed data and/or data generated by the processing module.

In addition, the electronic box according to the invention includes a first communication means 140, 141, 142 configured so that the electronic box 100 of at least one of the soles is adapted to transmit at least part of the transformed data to an external terminal 20. This data can be transmitted in real time or in delayed mode to an external terminal 20. The external terminal 20 can for example be a remote system such as a tablet, a mobile phone (“smartphone” in Anglo-Saxon terminology), a computer or a server.

Advantageously, each electronic box further includes a second communication means configured so that the electronic box 101 of a first sole is adapted to communicate with the electronic box 102 of a second sole, and so that at least one data processing module 121, 122 is configured to calculate, preferably jointly, data sets generated from the two soles 11,12, and more particularly from the inertial platform, constituting the pair 10 of soles.

The first and second communication means are adapted to receive and transmit the data over at least one communication network. Preferably, the communication is operated via a wireless protocol such as WiFi, 3G, 4G, and/or Bluetooth.

In addition, the electronic box according to the invention includes a power source 150, 151, 152. The power source is preferably of the battery type, rechargeable or not. Preferably, the power source is a rechargeable battery. In addition, it can be combined with a system for recharging by movement or with external energy. In particular, the system for recharging with external energy can be a wired recharging system, or an induction recharging system.

In addition, the electronic box according to the invention may include a wired connection means 160, preferably protected by a removable tab. This wired connection can be for example a USB or FireWire port. This wired connection means can be used as mentioned above to recharge the battery but also to exchange data and for example to update the firmware of the electronic board carrying the various components of the electronic box.

These various components of the electronic box are preferably arranged on an electronic board 170 (or printed circuit). In addition, the various means and modules of the electronic box 100 are shown separately in FIGS. 1 and 2, but the invention may provide for various types of arrangement such as, for example, a single module combining all the functions described here. Similarly, these means can be divided into several electronic boards or gathered on a single electronic board.

In addition, the system 1 includes an external terminal 20 adapted to receive data. The external terminal 20 is usually a tablet, a mobile phone (“smartphone” in Anglo-Saxon terminology), a computer, a server, or an autonomous on-board device. It may be adapted to transfer this data to a remote server 30. It is then possible, for example, to access this remote server via a web interface.

Advantageously, a dedicated application is installed on this external terminal in order to process the information transmitted by the boxes and allow the user to interact with the invention.

According to one embodiment, the invention could integrate a single box in one of the two soles. Indeed, this single box would communicate directly with the cyclist's external terminal.

As pedaling is a symmetrical movement of the lower limbs, it is possible to anticipate the biomechanical parameters of one of the two limbs by retrieving the biotechnical parameters of the other limb.

Since this invention can be related to the field of cyclist's accessories, it should be noted that, according to another embodiment, the boxes can be directly installed on the pedal cleats of a same bicycle or on any other device positioned at the soles, for example under the soles. In this hypothesis, the cyclist's biomechanical data is collected directly from the action exerted by his/her feet on each of the two pedals of the bicycle.

According to another embodiment, the two electronic boxes can be configured so as to transmit all the data collected to an autonomous on-board device carried by the cyclist, fixed to the bicycle frame, or simply positioned at the soles. Said autonomous on-board device comprising:
a communication module configured to receive the generated sets of data transmitted by the two electronic boxes, and
a data storage module configured to store the generated sets of data received,
   the communication module being further configured to transmit the generated sets of data received to an analysis device; and
an analysis device configured to process the collected sets of data according to predefined algorithms and generate information on the pedaling technique of a user of the pair of soles;
one or more force transducers, anemometers, strain gauges, or any other sensors which, combined with the boxes, would provide more information on the user's pedaling technique.

Such a system allows the user's pedaling to be reliably monitored. Indeed, the presence of a pair of soles, each including a box protecting an inertial platform, makes it possible to monitor the movement of each of the feet independently. The inertial platform will analyze, in at least three dimensions, the posture, movements, and cadence of the user's feet, and more generally everything that will be qualified as his/her pedaling. The inertial platform will be able to observe the different movements and postures of the feet, but also to identify weaknesses in the user's pedaling technique.

1. A system for analyzing and quantifying pedaling of a user, comprising two electronic boxes, adapted to be integrated into a pair of soles, a first said electronic box being adapted to be integrated into a first sole of said pair of soles and a second said electronic box being adapted to be integrated into a second sole of said pair of soles, each said electronic box comprising:
an inertial platform configured to generate a set of data on a pedaling technique of a user of the pair of soles,
a data processing module configured to, on the one hand, process the set of data generated according to predefined algorithms and transform the set of data into at least one biomechanical parameter and, on the other hand, determine an evolution of the at least one biomechanical parameter relating to the user's pedaling technique over time,
a data storage module configured to store information generated by the processing module, said information comprising said at least one biomechanical parameter, and
a power source;
wherein at least one of said two electronic boxes further comprises a first communication means adapted to transmit the information generated by the data processing module thereof to an external terminal and/or to the other of said two electronic boxes.

2. The system according to claim 1, wherein each said electronic box further includes a second communication means configured so that the first electronic box is adapted to communicate with the second electronic box, and so that at least one of the data processing modules is configured to transform data sets generated from the two electronic boxes into at least one biomechanical parameter relating to the pedaling technique.

3. The system according to claim 1, wherein the data processing modules communicate with each other and are adapted, on the one hand, to calculate an asymmetry between the biomechanical parameters of the right leg with respect to the biomechanical parameters of the user's left leg and, on the other hand, to calculate a variability of the biomechanical parameters associated with one leg of said user or both legs of said user.

4. The system according to claim 1, wherein at least one said data processing module is adapted to establish a pedaling technique profile of the user from biomechanical parameters selected from among foot angle, efficiency of push and pull phases of pedaling, foot position, cadence, rotational speed, and power.

5. The system according to claim 1, wherein the transformation by the data processing module comprises segmentation of the data into a plurality of pedaling phases.

6. The system according to claim 1, wherein each said electronic box further includes other sensors selected from among a magnetometer, a barometer, an altimeter, force and pressure sensors.

7. The system according to claim 4, wherein calculation of pedaling power is carried out following the combination of inertial and pressure/force data.

8. The system according to claim 1, wherein each of the two electronic boxes is adapted to be integrated into a pedal of a bicycle, or into a cleat of the pedal of a bicycle.

9. A method for quantifying a pedaling technique of a user implementing a quantification system including a pair of soles and an external terminal, each of the pair of soles including an electronic box, each said electronic box comprising an inertial platform, a data processing module, a data storage module, a first communication means, and a power source, said method including the following steps:
Generating, by the inertial platforms, a set of data on the pedaling technique of the user of the pair of soles,
Transforming, by the data processing modules, the generated data set into at least one biomechanical parameter, and comparing different values of the at least one biomechanical parameter,
Storing, by the data storage modules, different values of said at least one biomechanical parameter, and
Transmitting, by the first communication means of at least one of the soles, the at least one said biomechanical parameter and/or a value representative of an evolution of the user's pedaling to the external terminal.
